(19) United States
(12) Patent Application Publication
(10) Pub. No.: US 2006/0089694 A1
(43) Pub. Date: Apr. 27, 2006

(54) Delivery system and method for pulmonary artery leads

(75) Inventors: Yongxing Zhang, Maple Grove, MN (US); Yunlong Zhang, Mounds View, MN (US); Eric Lovett, Mendota Heights, MN (US); Bruce A. Tockman, Scandia, MN (US)

Correspondence Address: JASON R. KRAUS;FAEGRE & BENSON, LLP, 2200 WELLS FARGO CENTER, 90 SOUTH SEVENTH STREET, MINNEAPOLIS, MN, 55402-3901, US

(73) Assignee: Cardiac Pacemakers, Inc., St. Paul, MN (US)

(21) Appl. No.: 10/970,265

(22) Filed: Oct. 21, 2004

(51) Int. Cl. A61N 1/05

(52) U.S. Cl. 607/122

(57) ABSTRACT

A delivery system and method for delivering a pulmonary artery lead into a pulmonary artery includes a delivery device having an inflatable balloon at a distal end. The device is inserted into the venous system, the balloon is inflated and the device is floated along a blood flow path within the venous system through the heart and into the pulmonary artery. The lead is delivered into the pulmonary artery using the device. In one embodiment, the device is a catheter that facilitates placement of an implantation catheter and/or a guide wire into the pulmonary artery for delivery of the lead. In another embodiment, the lead is mounted over the catheter. In yet another embodiment, the delivery device is formed at the distal end of the lead.

### CROSS REFERENCE TO RELATED APPLICATIONS

This application is related to co-pending and co-owned application entitled DELIVERY SYSTEM AND METHOD USING PULMONARY ARTERY FOR PLACEMENT OF RV LEADS, filed on the same day and assigned Ser. No. ______, which is herein incorporated by reference.

### TECHNICAL FIELD

The present invention is related to the field of medical leads and related delivery systems and, in particular, to a delivery system for pulmonary leads.

### BACKGROUND

Cardiac leads have been placed in various locations within the heart structure in order to provide access for monitoring heart function and providing electrical stimulus to affect heart rhythm. One area of placement for such cardiac leads is the pulmonary artery. Pulmonary artery leads may be used for example for brady, tachy or AF/AT therapy, or for septal pacing and CHF therapy. Examples of pulmonary artery leads which provide these functions may be found in the following co-owned and co-pending patent applications: U.S. patent application Ser. No. 10/325,659 entitled PULMONARY ARTERY LEAD FOR ATRIAL THERAPY, filed Dec. 19, 2002; U.S. patent application Ser. No. 10/325,433 entitled IMPLANTABLE LEAD FOR SEPTAL PLACEMENT OF PACING ELECTRODES, filed Dec. 19, 2002; U.S. patent application Ser. No. 10/325,658 entitled IMPLANTABLE LEAD FOR SEPTAL PLACEMENT OF ELECTRODE WITH FIXATION MECHANISM IN THE PULMONARY ARTERY, filed Dec. 19, 2002; U.S. patent application Ser. No. 10/______ entitled CARDIAC LEAD FOR RA PACING/SENSING AND SHOCKING AND AF DEFIBRILLATING VIA THE PULMONARY ARTERY, filed Jul. 21, 2004; and U.S. patent application Ser. No. 10/______ entitled SINGLE-PASSAGE LEADS FOR BRADY AND TACHY THERAPIES: WITH SEPTAL/OUT FLOW TRACT PACING, RA PACING, SVC AND RV/SEPTAL SHOCKING, AND WITH FIXATION IN THE PULMONARY ARTERY, filed Jul. 21, 2004. Each of the above listed patent applications is hereby incorporated by reference.

Placement of pulmonary artery leads can be difficult and time consuming. There is still a need for a delivery system for pulmonary artery leads that provides accurate and efficient delivery of a lead into the pulmonary artery without, or with minimal, use of a fluoroscope.

### SUMMARY

The present invention is a delivery system and method for delivering a pulmonary artery lead into a pulmonary artery and includes a delivery device having an inflatable balloon at a distal end. The device is inserted into the venous system, the balloon is inflated and the device is floated along a blood flow path within the venous system through the heart and into the pulmonary artery. The lead is delivered into the pulmonary artery using the device. In one embodiment, the device is a catheter that facilitates placement of an implantation catheter and/or a guide wire into the pulmonary artery for delivery of the lead. In another embodiment, the lead is mounted over the catheter. In yet another embodiment, the delivery device is formed at the distal end of the lead.

While multiple embodiments are disclosed, still other embodiments of the present invention will become apparent to those skilled in the art from the following detailed description, which shows and describes illustrative embodiments of the invention. As will be realized, the invention is capable of modifications in various obvious aspects, all without departing from the spirit and scope of the present invention. Accordingly, the drawings and detailed description are to be regarded as illustrative in nature and not restrictive.

### BRIEF DESCRIPTION OF THE DRAWINGS

FIG. 1 shows a prior art pulmonary artery lead implanted into the pulmonary artery.

FIG. 2 shows a detailed view of a prior art pulmonary lead having internal lumen.

FIG. 3 shows one embodiment of a delivery system in accordance with the present invention for delivering a pulmonary lead into the pulmonary artery.

FIG. 4 shows a guide wire positioned in the pulmonary artery over which a pulmonary lead is delivered.

FIG. 5 shows a guide wire positioned in the pulmonary artery over which an implantation catheter is delivered.

FIG. 6 shows the implantation catheter of FIG. 5 through which a pulmonary lead is delivered.

FIG. 7 shows another embodiment of a delivery system in accordance with the present invention for delivering a pulmonary lead into the pulmonary artery.

FIG. 8 shown yet another embodiment of a delivery system in accordance with the present invention for delivering a pulmonary lead into the pulmonary artery.

### DETAILED DESCRIPTION

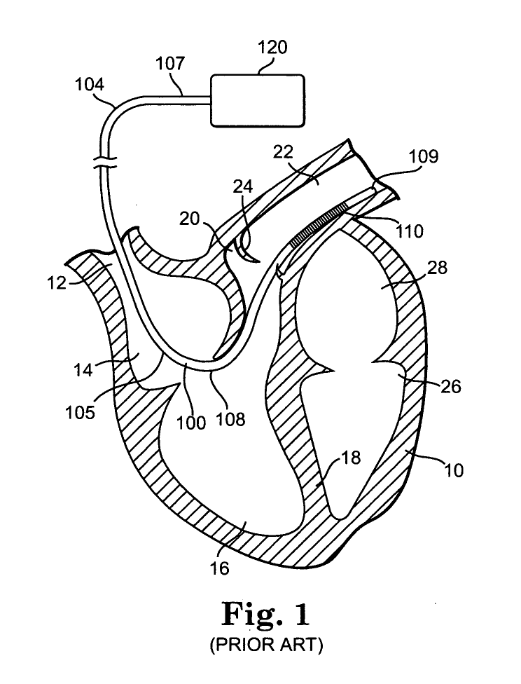

FIG. 1 shows a view of a lead 100 implanted within a heart 10. The heart 10 generally includes a superior vena cava 12, a right atrium 14, a right ventricle 16, a ventricular septum 18, a ventricular outflow tract 20, which leads to a pulmonary artery 22 having a pulmonary artery valve 24, a left ventricle 26 and a left atrium 28. In one embodiment, the lead 100 is adapted to deliver defibrillation pulses to the heart 10 via an electrode 110 positioned in the pulmonary artery 22. In this embodiment, the lead 100 is part of an implantable system including a pulse generator 120, such as a defibrillator. Alternatively, the lead 100 may be adapted to position electrodes adjacent to the ventricular septum, the right ventricle or ventricular outflow tract, or other suitable areas of the heart 10.

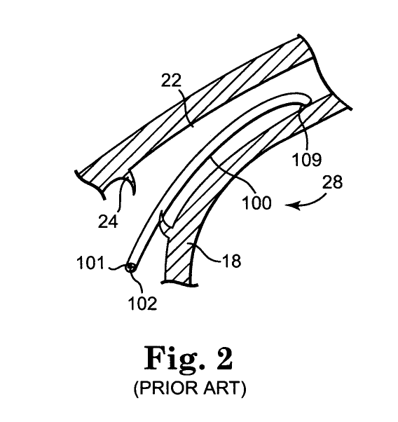

The lead 100 includes a lead body 105 that extends from a proximal end 107 to a distal end 109 and has an intermediate portion 108. The lead 100 includes one or more conductors, such as coiled conductors, to conduct energy, such as from pulse generator 120 to heart 10, and also to receive signals from the heart 10. The lead 100 further includes outer insulation 104 to insulate the conductor. The conductors are coupled to one or more electrodes, such as electrode 110. The lead 100 may also include one or more internal lumens 101, 102, as shown in FIG. 2.

In one embodiment, the lead 100 can be designed for placement of electrode 110 within the pulmonary artery 22 to deliver pulses, such as shock pulses, to the left atrium 28. The lead 100 is adapted for pulmonary artery placement of electrode 110, while utilizing pulmonary artery 22 for lead fixation. For example, in one embodiment, electrode 110 is coupled proximate the distal end 109 of the lead 100, such that placement of the distal end 109 into the pulmonary artery 22 results in corresponding placement of the electrode 110 into the pulmonary artery 22. Once the electrode 110 is in the desired location, the lead 100 may be fixed in place using one of many known techniques. These techniques include passive fixation by pre-formation of a bias shape at the distal end 109 of the lead 100, such as an S-shape, C-shape, J-shape, O-shape, spiral or other non-linear shape, as is known in the art. Alternatively, an active fixation mechanism may be provided, as is also known in the art. Other types of leads, as described above, may have different placement requirements within the pulmonary artery.

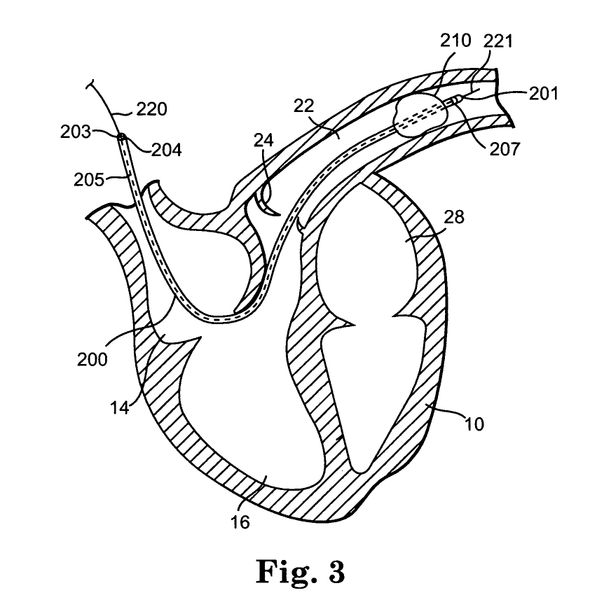

The present invention provides a delivery system for delivering a pulmonary artery lead to the pulmonary artery in an easy and efficient manner using a delivery device. In FIG. 3, a first embodiment of the delivery system includes a catheter 200 having a flexible member 205 with an inflatable balloon 210 positioned at a distal end 201. The catheter 200 includes at least a guide wire lumen 203 and an inflation lumen 204. The guide wire lumen 203 may be centered or may be off center and extends from a proximal end to the distal end 201. The inflation lumen 204 may also extend from a proximal end to the balloon 210 or, alternatively, it may branch from the catheter 200 at a proximal end to facilitate easier inflation of the balloon 210.

In one embodiment, the catheter 200 is similar to the Swan Ganz catheters described in U.S. Pat. No. 3,995,623, which is herein incorporated by reference. The catheter 200 is placed within a vein in a conventional manner and, when the catheter 200 is advanced into the right atrium 14 or beyond, then the balloon 210 is inflated allowing the balloon 210 and attached flexible catheter 200 to flow through the venous system into the heart 10 and out to the pulmonary artery 22. Further inflation of the balloon 210 affixes the catheter 200 temporarily within the pulmonary artery 22.

The catheter 200 is formed from a bio-compatible material, such as a flexible bio-compatible polymer, as is now known or later developed in the industry. In a similar manner, the balloon 210 is also formed from a bio-compatible material, such as thin, flexible latex or other suitable material. The catheter 200 may be sized as an 8 French or smaller.

A radiopaque marker 207 may optionally be provided on the distal end 201 of the catheter 200. This marker 207 may be used with a fluoroscopic or radiographic device to monitor the location of the catheter 200 within the venous system.

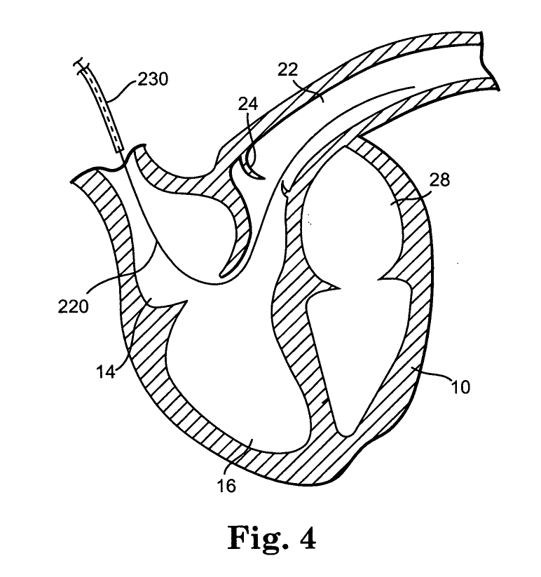

Once the catheter 200 is located within the pulmonary artery 22, a guide wire 220 may be inserted into the guide wire lumen 203 of the catheter 200 until a distal portion 221 of the guide wire 220 is also positioned within the pulmonary artery 22. When the guide wire 220 is in place, the balloon 210 may be deflated and the catheter 200 may be removed, as shown in FIG. 4.

A pulmonary lead 230, such as, for example, the leads described in the above listed co-owned and co-pending applications, may then be delivered to the pulmonary artery 22 by use of the guide wire 220. The lead 230 may be placed over the guide wire 220 using a conventional “over the wire” method to move the lead 230 into the pulmonary artery 22, as shown in FIG. 4. Once positioned within the artery 22, the lead 230 may be fixed using known techniques. The guide wire 220 is then removed from the heart 10 leaving the lead 230, similar to that shown in FIG. 1.

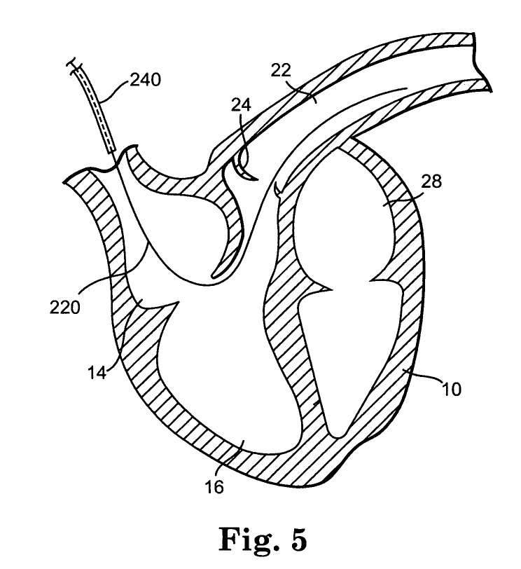

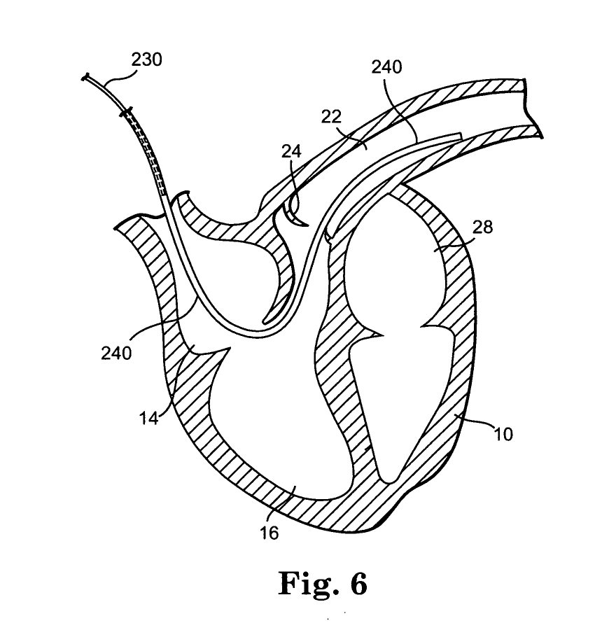

In a second embodiment of the delivery system of the present invention, instead of inserting a pulmonary lead, such as lead 230, over the guide wire 220, a lead implant catheter 240 is inserted over the wire 220, as shown in FIG. 5. Once in the lead implant catheter is in place, the guide wire 220 is removed. The pulmonary lead 230 is then delivered to the pulmonary artery 22 through the lead implant catheter, as shown in FIG. 6. When the lead 230 is fixed within the pulmonary artery 22, the lead implant catheter is removed from the heart 10 leaving the lead 230, similar to that shown in FIG. 1.

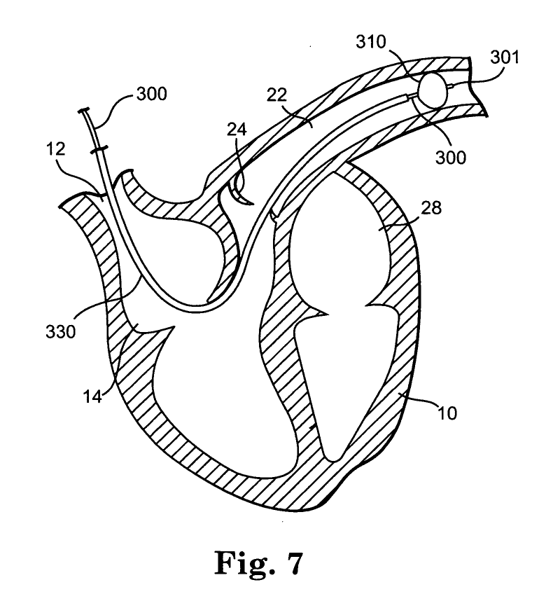

A third embodiment of the delivery system of the present invention is shown in FIG. 7. A catheter 300, similar to a Swan Ganz catheter, includes a balloon 310 at a distal end 301. The catheter 300 is configured to be inserted into the venous system and floated with the blood flow into and through the heart 10 out to the pulmonary artery 22. In this embodiment, a pulmonary lead 330 is positioned over the catheter 300 prior to insertion into the body or prior to floatation into the heart 10. As the catheter 300, led by the balloon 310, floats into the pulmonary artery 22, the lead 330 is carried with it, thereby also moving the lead 330 into the pulmonary artery 22. Once the pulmonary lead 330 is positioned appropriately, the balloon 310 is deflated and the catheter 300 is removed from the heart 10. The pulmonary lead 330 is then implanted or fixed in the pulmonary artery 22, as previously described.

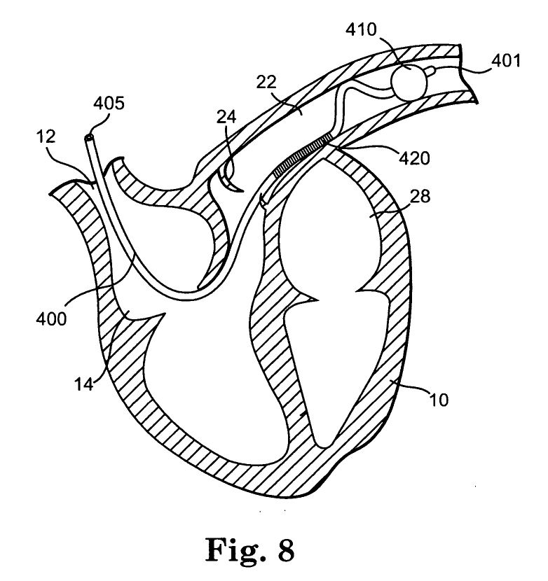

A fourth embodiment of the delivery system of the present invention is shown in FIG. 8. In this embodiment, a pulmonary artery lead 400, such as those described above, has an electrode 420 to be positioned in the pulmonary artery 22. The lead 400 is provided with an inflatable balloon 410 at a distal end 401 and an inflation lumen 405 for controlling the inflation and deflation of the balloon 410. Thus, instead of being delivered via a catheter, guide wire or both, the lead 400 may be inserted into the venous system and floated by blood flow into and through the heart 10 to the pulmonary artery 22. Once located within the artery 22, a guide wire or stylet may be used to adjust the lead 400 and place the electrode 420 in a desirable location. When finished, the balloon 410 is deflated and fixation of the lead 400 is completed.

Although the present invention has been described with reference to exemplary embodiments, persons skilled in the art will recognize that changes may be made in form and detail without departing from the spirit and scope of the invention.

We claim:

1. A method of delivering a cardiac lead into a pulmonary artery, the method comprising the steps of:
providing a delivery device having a distal end adapted for insertion into a venous system, the delivery device including an inflatable balloon positioned near the distal end;
inserting the delivery device at the distal end into the venous system;
inflating the balloon while within the venous system;
floating the balloon and delivery device along a blood flow path within the venous system through a heart and into the pulmonary artery; and
delivering the cardiac lead into the pulmonary artery using the delivery device.

2. The method of claim 1, further comprising the step of fixing the cardiac lead within the pulmonary artery.

3. The method of claim 1, further comprising the step of removing the delivery device from the pulmonary artery after delivery of the cardiac lead into the pulmonary artery.

4. The method of claim 1, wherein the step of inflating the balloon occurs when the delivery device is within a right atrium.

5. The method of claim 1, wherein the delivery device comprises a catheter and wherein the step of delivering the cardiac lead comprises the steps of:
inserting a guide wire through the catheter into the pulmonary artery;
removing the catheter from the pulmonary artery;
delivering the cardiac lead over the guide wire into the pulmonary artery; and
removing the guide wire from the pulmonary artery.

6. The method of claim 1, wherein the delivery device comprises a catheter and wherein the step of delivering the cardiac lead comprises:
inserting a guide wire through the catheter into the pulmonary artery;
removing the catheter from the pulmonary artery;
inserting a lead implantation catheter over the guide wire into the pulmonary artery;
removing the guide wire from the pulmonary artery;
delivering the cardiac lead using the lead implantation catheter into the pulmonary artery; and
removing the lead implantation catheter from the pulmonary artery.

7. The method of claim 1, wherein the delivery device comprises a catheter and wherein the step of delivering the cardiac lead comprises mounting the cardiac lead over the catheter prior to insertion into the venous system and floating the cardiac lead along with the catheter into the pulmonary artery.

8. The method of claim 7, further comprising the step of removing the catheter from the pulmonary artery.

9. The method of claim 1, wherein the delivery device comprises a distal portion of the cardiac lead such that floating the delivery device into the pulmonary artery includes delivering the cardiac lead into the pulmonary artery.

10. The method of claim 9, further comprising the step of adjusting the cardiac lead into a desirable location within the pulmonary artery.

11. The method of claim 10, wherein the step of adjusting includes using a guide wire or stylet to adjust the lead.

12. The method of claim 9, further comprising the step of deflating the balloon.

13. The method of claim 1, wherein the delivery device further comprises a radiopaque marker and further comprising the step of monitoring the location of the delivery device within the venous system by fluoroscopic monitoring of the radiopaque marker.

14. A delivery system for delivering a cardiac lead into a pulmonary artery, the delivery system comprising:
a delivery device having a distal end adapted for insertion into a venous system, the delivery device including an inflatable balloon positioned near the distal end; and
a cardiac lead configured for delivery into the pulmonary artery by use of the delivery device,
such that insertion of the delivery device into the venous system and subsequent inflation of the balloon results in floatation of the delivery device along a blood flow path within the venous system through a heart and placement of the delivery device into the pulmonary artery.

15. The delivery system of claim 14, wherein the cardiac lead comprises lead fixation structure for fixing the lead within the pulmonary artery.

16. The delivery system of claim 14, wherein the delivery device comprises a distal portion of the cardiac lead, such that floatation of the delivery device into the pulmonary artery also results in floatation of the cardiac lead into the pulmonary artery.

17. The delivery system of claim 14, wherein the delivery device comprises a catheter configured to receive a guide wire, such that placement of the catheter into the pulmonary artery facilitates placement of the guide wire into the pulmonary artery.

18. The delivery system of claim 17, wherein the cardiac lead is configured for delivery into the pulmonary artery by use of the guide wire.

19. The deliver system of claim 17, wherein the cardiac lead is configured for delivery into the pulmonary artery by use of a lead implantation catheter placeable into the pulmonary artery using the guide wire.
